(19) United States
(12) Patent Application Publication
(10) Pub. No.: US 2024/0309416 A1
(43) Pub. Date: Sep. 19, 2024

(54) A GENETICALLY ENGINEERED BACTERIUM AND ITS APPLICATION IN THE PREPARATION OF SIALYLLACTOSE

(71) Applicant: SYNAURA BIOTECHNOLOGY (SHANGHAI) CO., LTD., Shanghai (CN)

(72) Inventors: Yan WU, Shanghai (CN); Jing TANG, Shanghai (CN); Shu WANG, Shanghai (CN); Zhenhua TIAN, Shanghai (CN); Fei YAO, Shanghai (CN); Miao LI, Shanghai (CN); Hong XU, Shanghai (CN); Chenxi HUANG, Shanghai (CN); Yurou LIU, Shanghai (CN)

(73) Assignee: SYNAURA BIOTECHNOLOGY (SHANGHAI) CO., LTD., Shanghai (CN)

(21) Appl. No.: 18/576,640

(22) PCT Filed: Oct. 12, 2022
(86) PCT No.: PCT/CN2022/124823

§ 371 Date: Jan. 4, 2024

(30) Foreign Application Priority Data
Nov. 30, 2021 (CN) 202111450746.9

(51) Int. Cl. C12P 19/26; C12N 1/20; C12N 9/10; C12N 9/12; C12N 9/16; C12N 9/90; C12N 15/70; C12P 19/18; C12R 1/19

(52) U.S. Cl. CPC C12P 19/26; C12N 1/205; C12N 9/1029; C12N 9/1081; C12N 9/1085; C12N 9/1096; C12N 9/1241; C12N 9/1294; C12N 9/16; C12N 9/90; C12N 15/70; C12P 19/18; C12R 2001/19; C12Y 203/01004; C12Y 204/99001; C12Y 204/99004; C12Y 205/01056; C12Y 206/01016; C12Y 207/07043; C12Y 207/09002; C12Y 301/03; C12Y 501/03014

(57) ABSTRACT

The invention discloses a genetically engineered bacterium and its application in the preparation of sialyllactose. The genetically engineered bacterium has an N-acetylneuraminic acid biosynthesis pathway, includes multiple copies of a gene neuB for encoding sialic acid synthase, and the gene neuB is initiated for expression by a strong promoter. Using the genetically engineered bacteria of the invention to produce sialyllactose has the advantages of high yield and low overall cost.

### CROSS-REFERENCE TO RELATED APPLICATION

This application is a U.S. National Phase Entry of International Application No. PCT/CN2022/124823 filed on Oct. 12, 2022, which claims priority to Chinese Patent Application No. 202111450746.9 filed on Nov. 30, 2021, which are hereby incorporated herein by reference in their entireties.

### TECHNICAL FIELD

The invention belongs to the field of bioengineering, in particular to a genetically engineered bacterium and its application in the preparation of sialyllactose.

### BACKGROUND

Human milk oligosaccharides (HMOs) are one of the components with high nutritional value in human milk. According to the characteristics of the monosaccharide composition and structure, HMOs can be classified into neutral fucosyl, neutral non-fucosyl, and sialic acid, etc. Among them, the proportion of sialylated HMOs (SHMOs) is higher than 20%, and the species of the sialylated HMOs are relatively abundant. 3′-Sialyllactose (3′-SL), 6′-Sialyllactose, (6′-SL), and Sialyllacto-N-tetraose a (LST-a), etc., which are abundant in HMOs, have functions such as immune regulation, helping brain development and regulation of gut microbiota. Therefore, sialic acid always plays an important role in the rapid brain growth at the infant early development stage and the development process of immune system. However, the content of sialic acids in infant formulas currently on the market is low. Therefore, it is necessary to provide sialic acids of sufficient quality and quantity to supplement the infant formulas and other nutritional compositions.

At present, there are chemical and enzymatic synthesis, biosynthesis and other methods for the production of sialyllactose (SL). However, there are many difficulties in the actual production process of chemical synthesis or enzymatic synthesis, for example, stereochemical control, formation of specific linking, availability of raw materials, etc. The method through microbial metabolic synthesis using synthetic biology technology is more economical and efficient compared with chemical synthesis and enzymatic synthesis. Studies have shown that sialic acid (SA) can be synthesized by biosynthesis using glucose as a substrate, and the most abundant sialic acid in the products of this method seems to be N-acetylneuraminic acid (NANA, NeuNAc, Neu5Ac). This method is simple, has low cost, and produces considerable yield.

CN111133112A disclosed that a Neu5Ac-producing strain is obtained by a method comprising inactivation of lacZ and araA genes, deletion of N-acetylneuraminic acid catabolism gene clusters nagAB and nanAEKT, deletion of wzxC-wcaJ genes, deletion of fucl and fucK genes, and integration of galETKM, lacY, cscBKAR, glmS, glmM, glmU, Gna1, slr1975, neuBC, and ppsA into Escherichia coli genome, and is used for the screening of sialyltransferase. Some sialyltransferases with sialyltransferase activity are screened out, and can be used for the preparation of 3′-SL and 6′-SL. However, the resulting yield is not high because these genes are all integrated into the genome.

### SUMMARY

The technical problem to be solved by the present invention is to provide a genetically engineered bacterium and its application in the preparation of sialyllactose in order to overcome the defects of low yield and high production cost of the synthesis of sialyllactose in the prior art. Using the genetically engineered bacterium of the present invention to produce sialyllactose has the advantages of high yield and low overall cost.

The inventors creatively knocked out genes related to the intracellular degradation of N-acetylneuraminic acid (Neu5Ac) in the wild-type strain BL21 (DE3) by CRISPR/Cas9 technology, thereby constructing a non-naturally occurring Escherichia coli strain for the production of Neu5Ac, in which an Neu5Ac or SL synthetic pathway containing at least one heterologous enzyme is constructed and the naturally occurring N-acetylneuraminic acid (Neu5Ac) catabolic pathway is selectively disabled. The resulting genetically engineered bacterium can synthesize Neu5Ac or SL by means of self-metabolism using a single cheap exogenous carbon source in the fermentation broth.

The present invention solves the above technical problems mainly through the following technical solutions.

The first technical solution of the present invention is: a genetically engineered bacterium having an N-acetylneuraminic acid biosynthesis pathway, wherein the genetically engineered bacterium contains multiple copies of a gene neuB for coding a sialic acid synthase, and the gene neuB is initiated for expression by a strong promoter.

Preferably, an N-acetylneuraminic acid catabolic pathway in the genetically engineered bacterium is disabled. The disabled pathway may be conventional in the art, and in the present invention, the purpose of the disability is preferably achieved by knocking out all or part of the genes in the N-acetylneuraminic acid catabolism pathway.

Said all or part of the genes may be one or more of a gene nanK encoding N-acetylmannosamine kinase, a gene nanE encoding N-acetylmannosamine-6-phosphate epimerase, and a gene nanA encoding N-acetylneuraminic acid aldolase.

Further, a LacZ encoding the lactose operon β-galactosidase in the genetically engineered bacterium may also be knocked out.

The multiple copies described in the first technical solution may be achieved by conventional means in the art, for example, by one or more means of replicon, multi-site integration in genome and insertion of an exogenous plasmid. In a preferred embodiment of the present invention, the multiple copies are achieved by means of insertion of an exogenous plasmid.

The strong promoter described in the first technical solution may be conventional in the art, preferably is a Tet promoter.

The genetically engineered bacterium described in the present invention preferably further comprises a gene neuB encoding sialic acid synthase, a gene slr1975 encoding N-acetylglucosamine 2-epimerase, a gene YqaB encoding N-acetylglucosamine-6-phosphate phosphatase, a gene Gna1 encoding glucosamine-6-phosphate acetyltransferase, a gene glmS encoding L-glutamine-D-fructose-6-phosphate transaminase, and a gene ppsA encoding phosphoenolpyruvate synthase.

Preferably, the gene neuB, the gene slr1975, the gene YqaB, the gene Gna1, the gene glmS and the gene ppsA are linked in tandem in a plasmid vector 1.

Among these, the genes in the plasmid vector 1 preferably meet one or more of the following conditionas that:
the GenBank accession number of the gene neuB is AF305571;
the GenBank accession number of the gene slr1975 is BAL35720;
the gene YqaB is from the BL21 genome;
the GenBank accession number of the gene Gna1 is NP_116637;
the nucleic acid sequence of the gene glmS is set forth in SEQ ID NO: 66;
the gene ppsA is from the BL21 genome.

The backbone of the plasmid vector 1 in the present invention is preferably pACYCDuet.

The genetically engineered bacterium described in the present invention preferably further comprises a gene encoding N-acylneuraminate cytidylyltransferase and a gene encoding sialyltransferase; the two genes are preferably linked in tandem in a plasmid vector 2.

Wherein, the N-acylneuraminate cytidylyltransferase is preferably the N-acylneuraminate cytidylyltransferase with NCBI accession number WP_003512903.1, and preferably comprises the nucleic acid sequence set forth in SEQ ID NO: 61.

The sialyltransferase in the present invention may be conventional in the art, preferably is α-2,6-sialyltransferase or α-2,3-sialyltransferase. Wherein, the α-2,6-sialyltransferase is preferably an enzyme with NCBI accession number BAF91416.1, and preferably comprises the nucleic acid sequence set forth in SEQ ID NO: 62. The α-2,3-sialyltransferase is preferably an enzyme with NCBI accession number AJC62560.1, and preferably comprises the nucleic acid sequence set forth in SEQ ID NO: 63.

The backbone of the plasmid vector 2 in the present invention is preferably pET28a.

In the present invention, the starting bacterium of the genetically engineered bacterium is preferably Escherichia coli BL21 (DE3).

The second technical solution of the present invention is: a method for producing N-acetylneuraminic acid, comprising: culturing the genetically engineered bacterium as described in the first technical solution.

The third technical solution of the present invention is: a method for producing sialyllactose by fermentation, comprising: using the genetically engineered bacterium as described in the first technical solution, adding lactose into a fermentation medium for fermentation, and extracting the sialyllactose from the fermentation broth.

Optionally, 3′-sialyllactose is obtained when the genetically engineered bacterium comprises a α-2,3-sialyltransferase gene; 6′-sialyllactose is obtained when the genetically engineered bacterium comprises α-2,6-sialyltransferase gene.

The fermentation medium in the present invention is preferably a TB medium which comprises 12 g/L of trypsin, 24 g/L of yeast extract, 4 mL/L of glycerol, 2.31 g/L of KH₂PO₄ and 12.54 g/L of K₂HPO₄.

In the method described in the third technical solution, preferably, culture is induced with IPTG when cultured to an OD value of 0.6 to 0.8.

In addition, 2 g/L MgSO₄·7H₂O, 20 g/L glycerol, 1 mL/L trace element stock solution and 5 g/L lactose are supplemented after the induction culture; the trace element stock solution preferably comprises 54.4 g/L Ferric ammonium citrate, 9.8 g/L MnCl₂·4H₂O, 1.6 g/L CoCl₂·6H₂O, 1 g/L CuCl₂·2H₂O, 1.9 g/L H₃BO₃, 9 g/L ZnSO₄·7H₂O, 1.1 g/L Na₂MoO₄·2H₂O, 1.5 g/L Na₂SeO₃ and 1.5 g/L NiSO₄·6H₂O.

In the present invention, the fermentation culture conditions are preferably culture at 30° C. with shaking at 250 rpm.

The numbers following the terms in the present invention, such as “1” and “2” in plasmid vector 1 and plasmid vector 2, have no actual meaning, but only distinguish the same terms.

On the basis of conforming to common knowledge in the art, the above preferred conditions can be combined arbitrarily to obtain preferred examples of the present invention.

The reagents and raw materials used in the present invention are all commercially available.

The positive progressive effects of the present invention lie in:

On the one hand, the genetically engineered bacterium obtained in the present invention can obtain a higher yield of sialyllactose, and the yield of sialyllactose obtained after 24 h ours of shaking flask fermentation can reach 2.5 to 3 g/L; on the other hand, during the fermentation, the genetically engineered bacterium can synthesize sialyllactose by means of self-metabolism using a single cheap exogenous carbon source.

### BRIEF DESCRIPTION OF THE DRAWINGS

FIG. 1 shows the mapping of lacZ-knockout validation.

FIG. 2 shows the mapping of pTargetF plasmid.

FIG. 3 shows the mapping of the expression plasmid SL006.

FIG. 4 shows the mapping of pET28a-neuB plasmid.

FIG. 5 shows the mapping of pTac-neuB plasmid.

FIG. 6 shows the mapping of pTet-neuB plasmid.

FIG. 7 shows the mapping of SL023 plasmid.

FIG. 8 shows the mapping of SL023-Tet plasmid.

FIG. 9 shows the mapping of SL023-Tac plasmid.

FIG. 10 shows the HPLC detection profile of Neu5Ac in fermentation broth when the expression of neuB was regulated by the Tet promoter.

FIG. 11 shows the detection profile of 6′-SL standard, with the peak time of 17.818 min.

FIG. 12 shows the HPLC detection profile of 6′-SL in fermentation broth when the expression of neuB was regulated by the Tet promoter.

FIG. 13 shows the SL037 plasmid map.

FIG. 14 shows the detection profile of 3′-SL standard, with the peak time of 15.049 min.

FIG. 15 shows the HPLC detection profile of 3′-SL in fermentation broth when the expression of neuB was regulated by the Tet promoter.

### DETAILED DESCRIPTION

The present invention is further illustrated by the Examples below, but the present invention is not limited to the scope of the described Examples.

In order to further illustrate the technical means adopted in the present invention and its effects, they are described in detail in combination with the figures and the preferred embodiments of the present invention. The experimental methods in which the specific conditions are not indicated in the following examples are selected according to conventional methods and conditions, or according to the product instruction.

BL21 (DE3) strain was purchased from Novagen Company, with the article number of 69450-M; Escherichia coli Trans 10 competent cell was purchased from Beijing TransGen Biotech Co., Ltd.; plasmid extraction kit and gel recovery kit were purchased from Sangon Biotech (Shanghai) Co., Ltd., and the SDS-PAGE kit was purchased from Shanghai Yamei Biomedical Technology Co., Ltd.

HPLC detection method of sialyllactose: Chromatographic column: Sepax HP-Amide (250×4.6 mm, 5 um). Buffer salts: 10 mM ammonium formate (pH 3.0). Mobile phase: acetonitrile:buffer salts=70:30. Flow rate: 1.0 mL/min; concentration: 1 mg/mL; detection wavelength: 210 nm; injection volume: 10 μl; column temperature: 35° C.

### Example 1 Construction of Basic Strain SLIS026

### 1.1 Construction of a Plasmid Comprising the Small Guide RNA (sgRNA) Required for the CRISPR/Cas9 Knockout System

The specific amplification of each fragment is performed with primers designed according to Table 3 (synthesized by Tsingke) using pTargetF plasmid (see FIG. 2 for the mapping) or BL21 genome as a template, and high-fidelity enzyme Primer Star Mix from Takala was used for PCR reaction. The reaction system was as follows:

TABLE 1
PCR amplification reaction system

| | Volume added into the | |
|---|---|---|
| Reagent | PCR reaction system | |
| cDNA | 1 | μl |
| Primer F | 1 | μl |
| Primer R | 1 | μl |
| PCR Mix | 12.5 | μl |
| ddH₂O | 9.5 | μl |

The PCR amplification procedure was as follows:

TABLE 2
PCR reaction program

| | | | Cycle |
|---|---|---|---|
| Temperature | Time | | number |
| 98° C. | Pre-denaturation for 3 min | | |
| 98° C. | Denaturation for 15 s | | |
| 55° C. | Annealing for 15 | | |
| 72° C. | extension at 0.1 kb/s | {close oversize brace} | 34 cycles |
| 72° C. | extension for 5 min | | |
| 12° C. | hold | | |

5 μl of the amplified product was subjected to 1% agarose electrophoresis to detect the amplification result. The target fragments were recovered by gel cutting using a gel recovery kit. The target fragments were ligated and recombined using a multi-fragment recombinase from NEB, and the ligation and recombination products were transformed into E. coli competent cells Trans 10. Sterilized LB liquid medium was added, shook and cultured with shaking at 250 rpm at 37° C. for 1 h ;

(2) Spots were picked to the LB solid plate with spectinomycin added in advance, and the plate was inverted overnight at 37° C.;

(3) After white single colonies have grown, the white single colonies was picked into centrifuge tubes containing 2 mL of LB liquid medium (containing 50 μg/mL spectinomycin), and were cultured with shaking at 180 rpm for 6 h ours at 37° C.;

(4) The bacterium liquid was subjected to PCR assay, and 500 μl of the positive bacteriuml liquid, which was verified to be positive, was sent to Tsingke Company for sequencing, and the remaining bacterium liquid was stored in 20% glycerol.

(5) The strains which were verified by sequencing to be correct, were expanded, and plasmid extraction was carried out by using a plasmid extraction kit from Sangon. The sgRNA plasmids comprising the BL21 genome were obtained, and named as pTargetF-ΔLacZ, pTargetF-ΔnanKE, and pTargetF-ΔnanA, respectively.

TABLE 3
Primer information for construction of lacZ, nanAKE-knockout sgRNA
plasmid

| | | | | SEQ | | Product |
|---|---|---|---|---|---|---|
| | | Primer | | ID | | Size |
| Plasmid | Gene | Name | Sequence | NO: | Template | (bp) |
| pTargetF- | pTargetF | GA001- | tgccgaccgtctagagtcgacctgcagaag | 1 | pTargetF | 2000 bp |
| ΔLacZ | back- | P1-F4 | cttag | | | |
| | bone 1 | GA001- | aacTGGCGTTACCCAACTTA | 2 | | |
| | | P1-R4 | ATCactagtattatacctaggactg | | | |
| | PAM1 | GA001- | tagtGATTAAGTTGGGTAACG | 3 | pTargetF | 150 bp |
| | | P1-F1 | CCAgttttagagctagaaatagcaag | | | |
| | | GA001- | gttccggaattcaaaaaaagcaccgactcg | 4 | | |
| | | P1-R1 | gtgcc | | | |
| | LF | GA001- | gctttttttgaattccggaacgggaaggcga | 5 | BL21 | 560 bp |
| | | LF | ctggagtg | | | |
| | | GA001- | ggtgcgggcctcgacggccagtgaatccgt | 6 | genome | |
| | | P1-LR | aatcatg | | | |
| | RF | GA001- | tcgactctagacggtcggcaaagaccagac | 7 | BL21 | 1800 bp |
| | | P1-RR | cgttc | | | |
| | | GA001- | ctggccgtcgaggcccgcaccgategccct | 8 | genome | |
| | | P1-RF | tc | | | |
| pTargetF- | Donor- | pT14-F2 | gctttttttgaattcgggaattccgcagacac | 9 | BL21 | 1000 bp |
| ΔnanKE | LF | | catctg | | | |
| | | pT-G08- | ccaaaagttaatgatgggtgaagtacagtca | 10 | genome | |
| | | R1 | ttac | | | |
| | Donor- | pT-G08- | gtacttcacccatcattaacttttggttttgac | 11 | BL21 | 1000 bp |
| | RF | F2 | | | | |
| | | pT-G08- | gtcgactctagagcgtgcgctgttctttatcg | 12 | genome | |
| | | R2 | g | | | |
| | pTargetF | pT-G08- | cagcgcacgctctagagtcgacctgcagaa | 13 | pTargetF | 1000 bp |
| | back- | F3 | g | | | |
| | bone | pT-G08- | acCACGCGTGGCTTGCAGAT | 14 | | |
| | | R3 | TTactagtattatacctag | | | |
| | PAM | pT-G08- | agtAAATCTGCAAGCCACGC | 15 | pTargetF | 150 bp |
| | | F1 | GTGgttttagagctagaaata | | | |
| | | pT4-1R1 | ctaaaacCGATCGTCGTGAACT | 16 | | |
| | | | TCCTAactagtattatacctagg | | | |
| pTargetF- | Donor- | pT15-F2 | gctttttttgaattcgcagcgcctgaatcggc | 17 | BL21 | 1000 bp |
| ΔnanA | LF | | catgag | | genome | |
| | | pT15- | gaggtatttgttgtttcccctcgctcgccccta | 18 | | 1000 bp |
| | | 1R2 | cc | | | |
| | Donor- | pT15- | gcgaggggaaacaacaaatacctctgaagt | 19 | BL21 | |
| | RF | 1F3 | gatgcttg | | genome | |
| | | pT15-R3 | caggtcgactctagaccgcccgctggcgc | 20 | | |
| | | | gtaaaaaac | | | |
| | PAM1 | pT15-P1- | actagtATCGACGGTTTATAC | 21 | pTargetF | 150 bp |
| | | F1 | GTGGGgttttagagctagaaatag | | | |
| | | pT15-R1 | gcgaattcaaaaaaagcaccgactcggtgc | 22 | | |
| | | | cac | | | |
| | pTargetF | pT15-F4 | gcgggcggtctagagtcgacctgcagaag | 23 | pTargetF | 2000 bp |
| | back- | | cttag | | | |
| | bone | pT15- | aacCCCACGTATAAACCGTC | 24 | | |
| | | 1R4 | GATactagtattatacctagg | | | |

### 1.2 Gene Knockout of lacZ and nanAKE

1.2.1 BL21 Strain lacZ (GA001) Gene Knockout

(1) Preparation of BL21 competent cells: the strain BL21 stored at −80° C. was streaked and cultured to obtain single colonies; the single colonies were picked up and inoculated in 5 mL of LB medium, and cultured with shaking at 200 rpm at 37° C. until the OD reaches about 0.5 (for about 3 h ), then the culture was ice-bathed for 30 min; the bacterial broth was transferred to a pre-cooled sterile centrifuge tube and centrifuged at 4000 rpm for 10 min at 4° C., then the supernatant was discarded, and the bacteria were collected; the bacteria were resuspended with pre-cooled sterile water and centrifuged at 4000 rpm for 10 min at 4° C., then the supernatant was discarded; the bacteria were resuspended with a solution containing 0.1M CaCl₂ twice, and centrifuged at 4000 rpm for 10 min at 4° C., then the supernatant was discarded; finally, the cells were resuspended with appropriate amount of 15% glycerol in 0.1M CaCl₂ solution, divided into 1.5 mL centrifuge tubes with 100 μl per tube, rapidly frozen in liquid nitrogen, and stored at −80° C.

(2) 3 μl pCas-sac plasmid was added to 100 μL E.coli BL21 competent, placed on ice for 30 min, then heat-shocked at 42° C. for 45 s, and immediately placed on ice for 2-5 min; placed on a shaker at 30° C. and incubated for 45 min after adding 800 μL LB, spread onto a plate (Km resistant, LB medium), inverted in an incubator at 30° C., and cultured overnight; the colony was picked out into LB medium (Kana resistant), and cultured for several hours followed by bacteria preservation (30% glycerol final concentration).

(3) The pCas-sac/BL21 transformants were picked and inoculated into LB sieve tubes (Kara-resistant), cultured at 30° C. until the OD reaches 0.2, and arabinose was added at a final concentration of 2 g/L for induction, and competent preparation was performed when the OD reaches 0.4, the preparation method was the same as operation (1);

(4) The correctly constructed pTargetF-ΔLacZ plasmid was transformed into pCas-sac/BL21 competent cells by heat shock method, spread onto LB plates (k+, spe+) after recovery, and cultured at 30° C. overnight;

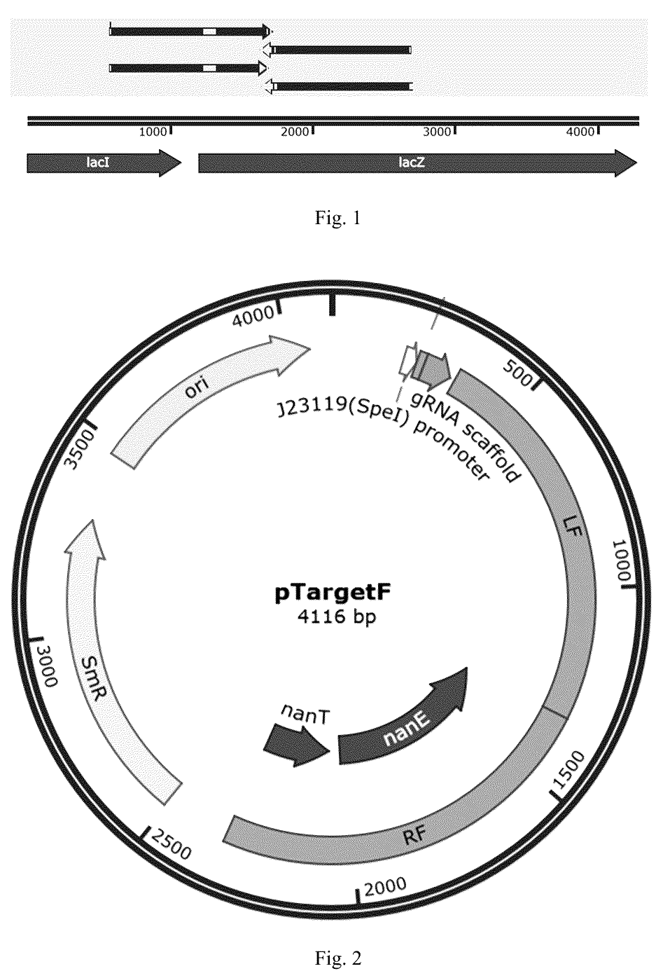

(5) PCR validation was carried out on single colonies on the resistant plate. The primers for validation are shown in Table 4, and the mapping of sequencing validation is shown in FIG. 1. The strain with LacZ gene knockout was obtained by verification;

(6) The strains with LacZ gene knockout were selected and cultured with shaking, and induced by adding rhamnose at a final concentration of 10 mM for the loss treatment of the sgRNA plasmid pTargetF-ΔLacZ;

(7) The pTargetF-ΔLacZ plasmid was streaked to verify whether the loss occurred (see Table 4 for primers), and the strain with sgRNA loss and LacZ gene knocked out was named as SLSI020.

1.2.2 Knockout of Neu5Ac Degradation Related Gene nanKAE Gene Based on SLIS020 Strain

(1) Competent preparation and knockout operation of SLIS020 were the same as 1.2.1. The pTargetF-ΔnanA plasmid was used to knock out the nanA gene, and the method was the same as 1.2.1, to obtain the strain with nanA gene knockout, which was named as SLIS024.

(2) Knockout of nanKE gene was performed on SLIS024 strain, using pTargetF-ΔnanE plasmid for knockout, the method was the same as 1.2.1, and the strain with nanKE gene knockout completed was named as SLIS026.

(3) The SLIS026 strain was subjected to loss of sgRNA plasmid, and the method was the same as 1.2.1.

(4) The SLIS026 strain was subjected to loss of pCas-SAC plasmid: the SLIS026 strain with sgRNA loss was inoculated on non-resistant LB plate, cultured at 42° C., and verified by PCR with pCas-SAC validation primers in Table 4 to ensure that the chassis strain SLIS026 without pCas-SAC plasmid was obtained.

TABLE 4
Primers for validation of LacZ, nanAKE and other gene knockouts

| | Knockout | | | SEQ ID | Product |
|---|---|---|---|---|---|
| Strain | gene | Primer Name | Sequence | NO: | Size (bp) |
| SLIS020 | lacZ | Lacz-YZ1-F | cgcgctgttagcgggcccattaagttctg | 25 | 2000 bp |
| | | LAC-YZ2-R | ggtcttcatccacgcgcgcgtacatcgg | 26 | after |
| | | | | | knockout |
| SLIS024 | NanA | nanA-YZ-F1 | catggtgatgtagcctggcgcaaagcc | 27 | 2.2 kb after |
| | | nanA-YZ-R | atgggccttatgaacgcatttgattcgc | 28 | knockout |
| SLIS026 | NanKE | nanE-YZ-F | gctcgtgcaattccgcttttttctcgac | 29 | 1.5 kb after |
| | | nanE-YZ-R | cggttatttcgataccgaccagcgtgcag | 30 | knockout |
| sgRNA | | CX-targetF-F | cagcgagtcagtgagcgag | 31 | 2000 bp |
| plasmid | | CX-targetF-R | gacattgcactccaccgct | 32 | |
| pCas-SAC | | Kan-F | gaaggagaaaactcaccgag | 33 | 3300 bp |
| | | Pcr4-R1 | cagctgcataaaattgcgattggcaaaa | 34 | |
| | | | ccatc | | |

### Example 2 Construction of Expression Plasmid Related to N-Acetylneuraminic Acid (Neu5Ac) Synthesis and Preparation of Neu5Ac

### 2.1 Construction of Expression Plasmid for Neu5Ac Synthesis

(1) The gene glmS* (GA010) is a mutant version of Escherichia coli L-glutamine-D-fructose-6-phosphate transaminase gene (Metab Eng., 2005 May; 7(3):201-14), and its nucleic acid sequence is set forth in SEQ ID NO: 66; Gna1 (GA009, GenBank:NP_116637) encodes the glucosamine-6-phosphate acetyltransferase from Saccharomyces cerevisiae; the gene slr1975 (GA006, GenBank: BAL35720) encodes Synechocystis sp. PCC6803 N-acetylglucosamine 2-epimerase; the gene neuB (GA005, GenBank:AF305571) encodes the sialic acid synthase from Campylobacter jejuni; the gene ppsA encodes the phosphoenolpyruvate synthase of Escherichia coli BL21 (DE3); the gene YqaB encodes the N-acetylglucosamine-6-phosphate phosphatase. The slr1975, Gnal, glmS, neuB, and promoter Tet, Tac gene sequences were synthesized and ligated into the puc57 vector by Sangon Biotech (Shanghai) Co., Ltd., and YqaB and ppsA were all from the BL21 genome.

(2) PCR amplification was performed according to the primers and templates listed in Table 5 to obtain the target fragments. The PCR reaction system and conditions are the same as 1.1 in Example 1.

(3) The amplified DNA fragments were recovered by gel cutting using a gel recovery kit, and positive single colonies were obtained by recombination, transformation, and plate screening using the multi-fragment recombination kit of NEB Company, and then the correct plasmids were obtained by picking up the colony, shaking bacteria, and verifying via sequencing by Tsingke Biotechnology Co., Ltd.

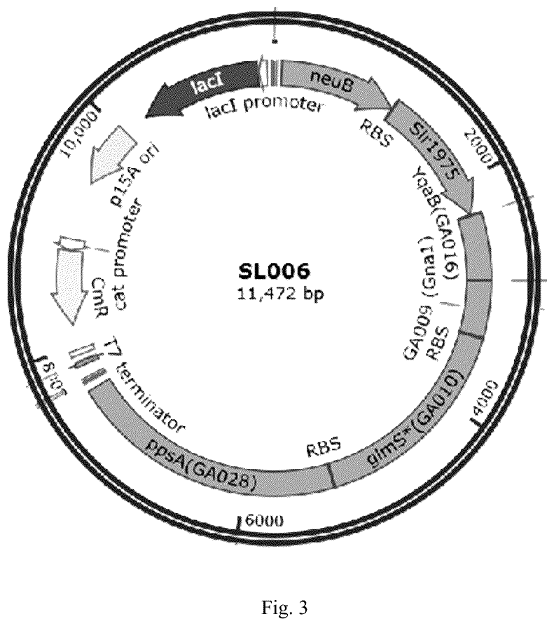

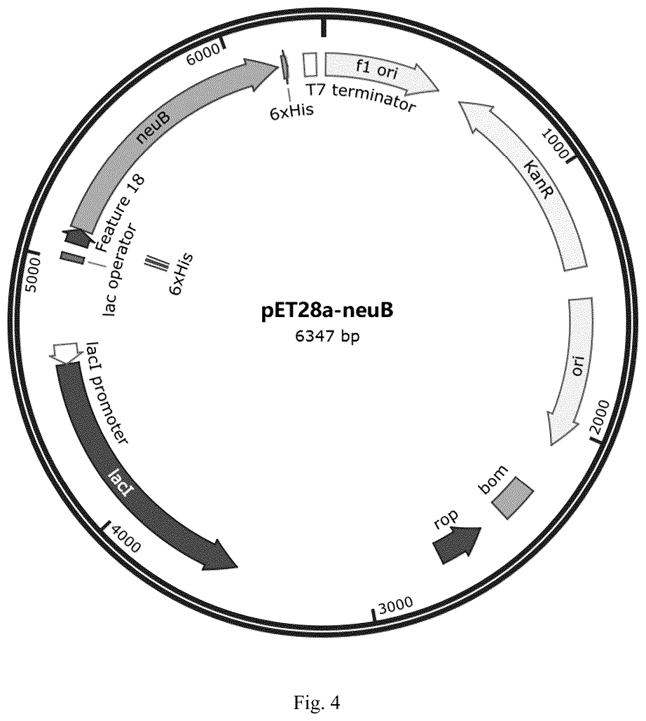

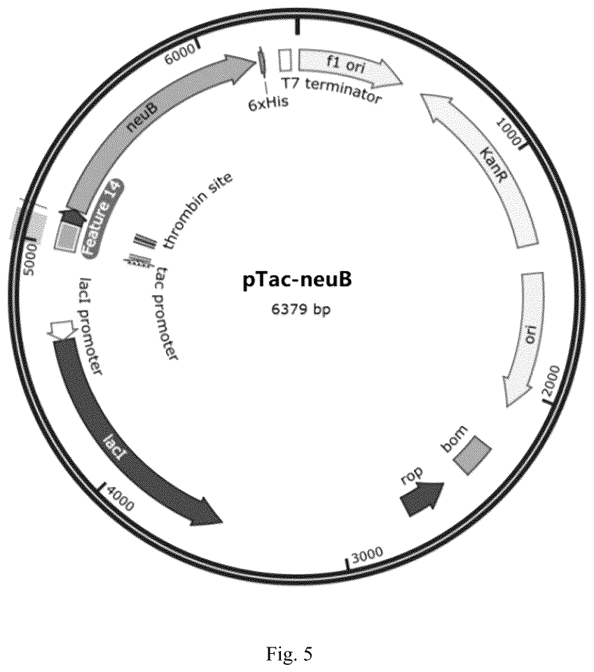

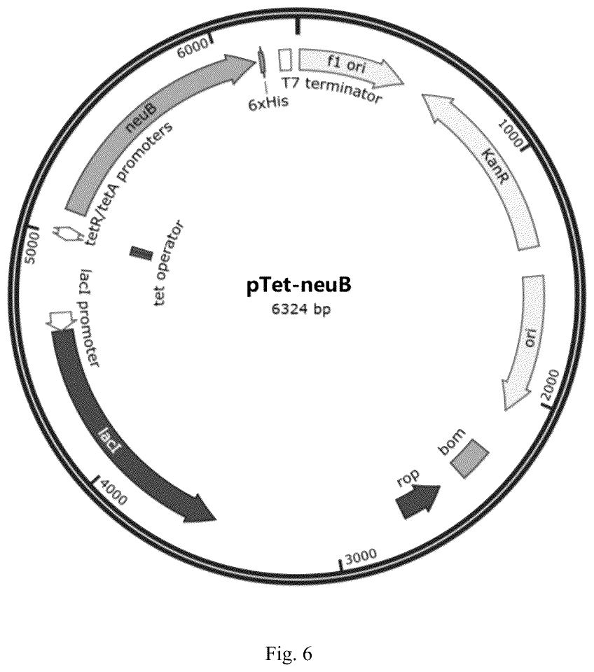

TABLE 5
Primers required for the plasmid construction of expression plasmids
SL006, pET28a-neuB and others

| | | Primer | | SEQ ID | | Product |
|---|---|---|---|---|---|---|
| Plasmid | Gene | Name | Primer Sequence | NO: | Template | Size |
| SL006 | neuB | ACY-GA5- | aataaggagatataatgaaagaaatcaa | 35 | GA005 | 1 kb |
| (FIG. 3) | | F1 | aatcc | | | |
| | | ACY-GA5-R | ggtgagcgatcatggtatatctccttttag | 36 | | |
| | | | gcgaaatcttcataag | | | |
| | slr1975 | ACY-GA6-F | accatgatcgctcaccgtcgtcaggaac | 37 | GA006 | 1.1 kb |
| | | | tgg | | | |
| | | ACY-GA6-R | gctcgtacatggtatatctccttttaagag | 38 | | |
| | | | accggcagttg | | | |
| | YqaB | ACY-GA16- | gatataccatgtacgagcgttatgcagg | 39 | BL21 | 567 bp |
| | | F | | | | |
| | | AVY-GA16- | cgggtaagctcatggtatatctcctttcac | 40 | genome | |
| | | R | agcaagcgaacatcc | | | |
| | Gnal | ACY-GA9-F | taccatgagcttacccgatggattttatat | 41 | GA009 | 480 bp |
| | | | aag | | | |
| | | ACY-GA9-R | cgataccgcacatggtatatctccttctatt | 42 | | |
| | | | ttctaatttgc | | | |
| | glms | ACY-GA10- | taccatgtgcggtatcgttggtgctatcg | 43 | GA010 | 1.8 kb |
| | | F | | | | |
| | | ACY-GA10- | gccattgttggacatggtatatctcctttta | 44 | | |
| | | R | ttccacggtcacggatt | | | |
| | ppsA | ACY-GA28- | ccatgtccaacaatggctcgtcaccgct | 45 | BL21 | 2.8 kb |
| | | F | ggtg | | | |
| | | ACY-GA28- | ccgcaagcttttatttcttcagttcagcca | 46 | genome | |
| | | R | gg | | | |
| | Back- | ACY-F | gaaataaaagcttgcggccgcataatgc | 47 | pACYC | 4 kb |
| | bone | PACY-R | gatttctttcattatatctccttattaaagtta | 48 | Duet | |
| | | | aac | | | |
| pET28a- | Back- | 28a-G5F2 | gatttcgcctaaGCTTGCGGCC | 49 | pET28a | 5 kb |
| neuB | bone | | GCACTCGAGCAC | | | |
| (FIG. 4) | | 28a-G5R2 | gatttctttcatATGGCTGCCGC | 50 | | |
| | | | GCGGCACCAGG | | | |
| | neuB | 28a-G5F1 | ctggattttgatttctttcatATGGCT | 51 | GA005 | 1 kb |
| | | | GCCGC | | | |
| | | 28a-G5R1 | CCGCAAGCttaggcgaaatcttca | 52 | | |
| | | | taagacagctg | | | |
| pTac- | Tac | Tac-F1 | GAAATttgacaattaatcatcggctc | 53 | Puc-Tac | 136 bp |
| neuB | promoter | | gtataatgtg | | | |
| (FIG. 5) | | Tac-R1 | GGCTGCTGCCCATgtatatct | 54 | | |
| | | | ccttcttaaagttaaac | | | |
| | neuB | Tac-F2 | gaaggagatatacATGGGCAGC | 55 | pET28a- | 6.29 kb |
| | | | AGCCATCATCATC | | neuB | |
| | | Tac-R2 | gatgattaattgtcaaATTTCGCG | 56 | | |
| | | | GGATCGAGATCTC | | | |
| pTet- | Tet | Tet-F1 | GATCCCGCGAAATgttgaca | 57 | Puc-Tet | 150 bp |
| neuB | promoter | | ctctatcattgatag | | | |
| (FIG. 6) | | Tet-R1 | tgatttctttcatttgtatatctccttcttaaa | 58 | | |
| | | | g | | | |
| | neuB | Tet-F2 | gatatacaaatgaaagaaatcaaaatcc | 59 | pET28a- | 6.29 kb |
| | | | agaac | | neuB | |
| | | Tet-R2 | gatagagtgtcaacATTTCGCGG | 60 | | |
| | | | GATCGAGATCTCG | | | |

(4) Promoter sequence:

The sequence of the Tac promoter is set forth in SEQ ID NO: 64.

The sequence of the Tet promoter is set forth in SEQ ID NO: 65.

### 2.2 Preparation of N-Acetylneuraminic Acid (Neu5Ac)

2.2.1 Construction of N-Acetylneuraminic Acid (Neu5Ac)-Producing E. coli Strains

Competent cells were prepared on the basis of the gene knockout strain SLIS026, and the specific method was the same as 1.2.1. Then plasmid SL006 were transformed, alone, and together with pET28a-neuB, pTac-neuB, and pTet-neuB, into SLIS026 competent cells, and the correct clones were screened on LB plates (kanamycin 50 μg/mL, chloramphenicol 25 μg/mL). Four strains E. coli SLIS026-SA (SL006, SL006+pET28a-neuB, SL006+pTet-neuB, SL006+pTac-neuB) carrying Neu5Ac synthesis pathway were obtained correspondingly after validation by PCR.

### 2.2.2 Production of N-Acetylneuraminic Acid (Neu5Ac) by SLIS026-Neu5Ac Strain

(1) TB medium: 12 g of trypsin, 24 g of yeast extract, 4 mL of glycerol, 2.31 g KH₂PO₄, 12.54 g K₂HPO₄, supplemented to 1000 mL with deionized water, and finally dispensed into conical flasks at 100 mL/bottle and wrapped well, sterilized at 121° C. for 30 min, and stored at room temperature.

(2) LB medium: 10 g of tryptone, 5 g of yeast extract, 10 g of NaCl, and 15 g of agar were weighed respectively, and were dissolved with distilled water and mixed well, and were supplemented to 1 L after the pH was adjusted to 7.2 with 1 mol/L NaOH, and finally divided into conical flasks and wrapped well, sterilized at 121° C. for 30 min, and stored at 4° C. The LB liquid was withour agar.

(3) 200 g/L MgSO₄·7H₂O stock solution: 10 g MgSO4·7H2O solution was weighed and dissolved into deionized water, supplemented to 5 mL after completely dissolved, then sterilized at 121° C. for 30 minutes, and stored at room temperature.

(4) Trace element stock solution: 54.4 g/L ferric ammonium citrate, 9.8 g/L MnCl₂·4H₂O, 1.6 g/L CoCl₂·6H₂O, 1 g/L CuCl₂·2H₂O, 1.9 g/L H₃BO₃, 9 g/L ZnSO₄·7H₂O, 1.1 g/L Na₂MoO₄·2H₂O, 1.5 g/L Na₂SeO₃, 1.5 g/L NiSO₄·6H₂O. The trace element stock solution was prepared according to the above concentrations, sterilized at 121° C. for 30 min, and stored at 4° C. for future use.

(5) 1000 g/L glycerol: 1000 g glycerol was weighed, supplemented to 1 L with deionized water, then sterilized at 121° C. for 30 minutes, and stored at room temperature.

(6) The strains were inoculated into 5 mL of LB medium (kanamycin 50 μg/mL, chloramphenicol 25 μg/mL), and cultured at 37° C. and 250 rpm for 4 h . Then the seed liquid was inoculated into fresh TB medium at a ratio of seed liquid: medium=1: 100, cultured at 37° C., 250 rpm to OD600=0.6-0.8, and IPTG was added to a final concentration of 0.1 mM and the medium was cultured under the condition of 25° C., 250 rpm for 15 h for the inducible expression of protein.

(7) After the inducible expression of protein, 1 mL of 200 g/L MgSO₄·7H₂O (final concentration of 2 g/L), 1 mL of trace element stock solution (final concentration 0.1%), and 2 mL of 1000 g/L glycerol (final concentration of 20 g/L) were added to the shaking flask, and cultured at 30° C. and 250 rpm for 24 h, and then sampled to detect the content of Neu5Ac.

(8) Means of sample treatment: the fermented bacterial solution was sterilized at 121° C. for 20 min, then centrifuged (4° C., 4000 rpm for 15 min) to remove the precipitation and retain the supernatant, and supplemented to 100 ml with the sterilized TB medium for future use. 1 mL of the solution was sampled and passed through a 0.22 μm filter membrane, and detected with ion-pair ion. The strains using the Tet promoter were detected to have the highest Neu5Ac yields, 2.9 g/L of Neu5Ac. FIG. 10 shows the Neu5Ac detection profile.

### Example 3 Construction of 6′-SL (6′-Sialyllactose) Synthesis-Related Expression Plasmid and Preparation of 6′-SL

### 3.1 Construction of 6′-SL Synthesis Plasmid

(1) Total gene synthesis was performed on the gene SEQ ID NO: 61 of N-acylneuraminate cytidylyltransferase CSS (accession number WP_003512903.1) (GA031); the gene SEQ ID NO: 62 of α-2,6-2,6-sialyltransferase (6ST, GA025) with accession number BAF91416.1, and both gene sequences were synthesized and ligated into the pET28a vector by Sangon Biotech (Shanghai) Co., Ltd.

(2) PCR amplification was performed according to the primers and templates listed in Table 6 to obtain the target fragments. The PCR reaction system and conditions were the same as 1.1 in Example 1.

(3) The amplified DNA fragments were recovered by gel cutting using a gel recovery kit, and positive single colonies were obtained by recombination, transformation, and plate screening using the multi-fragment recombination kit of NEB Company, and then the correct plasmids were obtained by picking up the colony, shaking bacteria, and verifying via sequencing by Tsingke Biotechnology Co., Ltd.

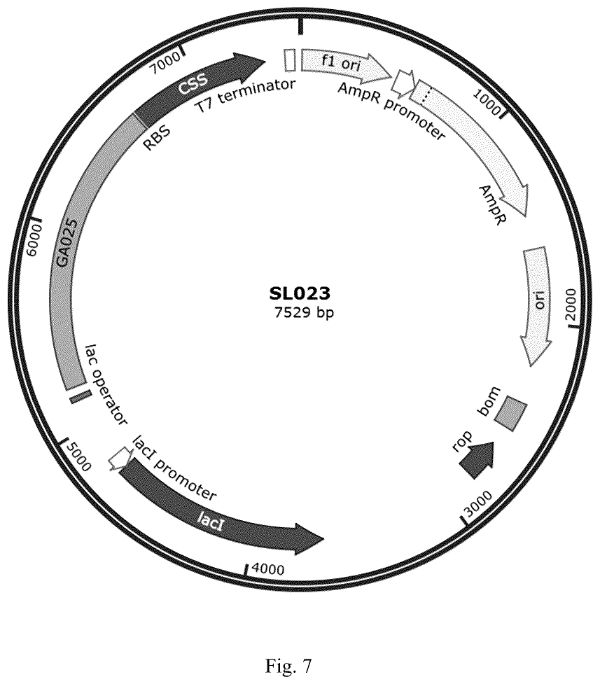

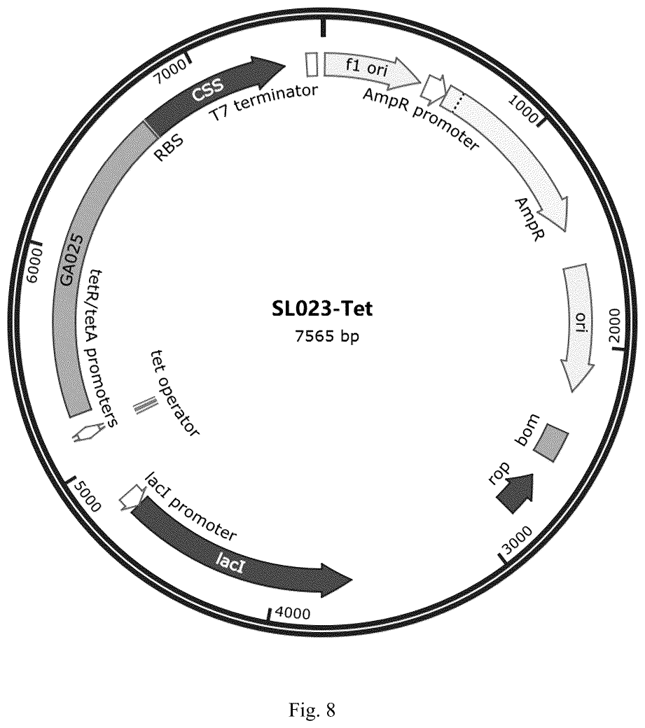

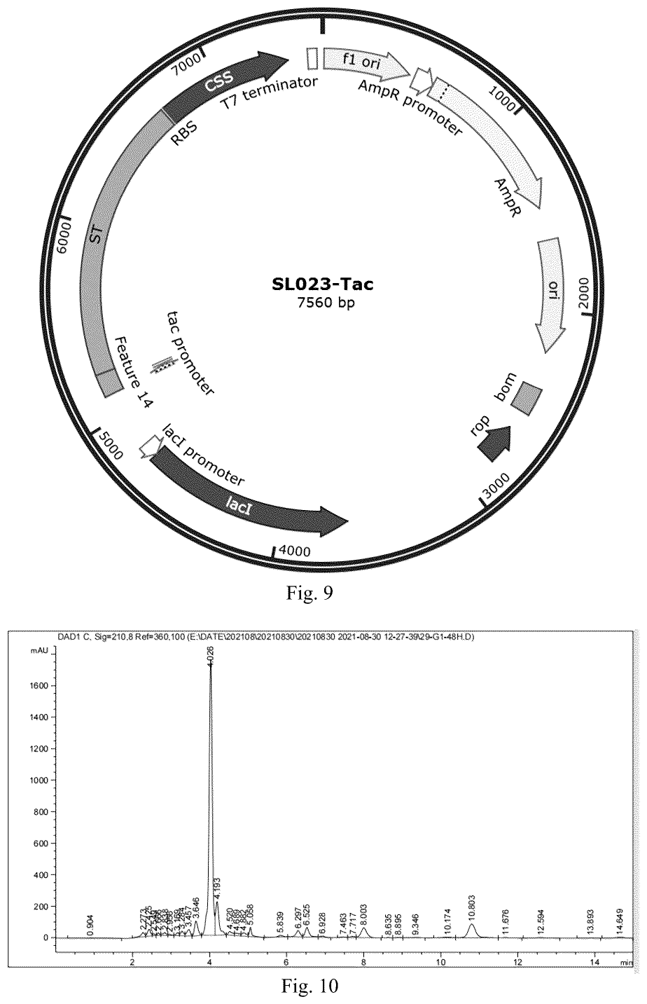

TABLE 6
Primers required for construction of expression plasmid SL023

| | | Primer | | SEQ ID | Temp- | Product |
|---|---|---|---|---|---|---|
| Plasmid | Gene | Name | Primer Sequence | NO: | late | Size |
| SL023 | 6ST | SL23-F1 | gatatacatatgaacgataatcaaaatacggtgg | 67 | GA025 | 1400 bp |
| (FIG. 7) | | SL23-R1 | CCATggtatatctccttttagcaccagaacagcac | 68 | | |
| | | | atctttttc | | | |
| | CSS | SL23-F2 | gtgctaaaaggagatataccATGGAAAAAC | 69 | GA031 | 687 bp |
| | | | AGAACATCGCGG | | | |
| | | SL023- | gtgcggccgcaagcttaAGATTCTTTGTG | 70 | | |
| | | R2 | GTTCAGGATG | | | |
| | F3 | SL023- | CAAAGAATCTtaagcttgcggccgcactcg | 71 | pET28a | 5.2 kb |
| | | F3 | agcacc | | | |
| | | SL23-R3 | gattatcgttcatatgtatatctccttcttaaag | 72 | | |
| SL023- | Tet | Tet-F1 | GATCCCGCGAAATgttgacactctatcatt | 73 | Puc- | 150 bp |
| Tet | promoter | | gatag | | Tet | |
| (FIG. 8) | | SL023- | tgattatcgttcatttgtatatctccttcttaaag | 74 | | |
| | | Tet-R1 | | | | |
| | Tet-F | SL23- | gatatacaaatgaacgataatcaaaatacggtgg | 75 | SL023 | 7.3 kb |
| | | Tet-F1 | | | | |
| | | Tet-R2 | gatagagtgtcaacATTTCGCGGGATCG | 76 | | |
| | | | AGATCTCG | | | |
| SL023- | Tac | Tac-F1 | cgcgaaatttgacaattaatcatcggctcgtataatgtg | 77 | Puc- | 141 bp |
| Tac | promoter | SL023- | tgattatcgttcatgtatatctccttcttaaag | 78 | tac | |
| (FIG. 9) | | Tac-R1 | | | | |
| | Tac-F | SL23- | gagatatacatgaacgataatcaaaatacggtgg | 79 | SL023 | 7.3 kb |
| | | Tac-F1 | | | | |
| | | Tac-R2 | gatgattaattgtcaaatttcgcgggatcgagatctcg | 80 | | |

### 3.2 Production of 6′-SL Using Strain SLIS026

3.2.1 Construction of 6′-SL E. coli Strains

Competent cells were prepared on the basis of the gene knockout strain SLIS026, and the specific method was the same as 1.2.1. Then the following plasmid combinations: SL006+pET28a-neuB+SL023, SL006+pET28a-neuB+SL023-Tet, SL006+pET28a-neuB+SL023-Tac, SL006+pTet-neuB+SL023, SL006+pTet-neuB+SL023-Tac, SL006+pTet-neuB+SL023-Tet, SL006+pTac-neuB+SL023, SL006+pTac-neuB+SL023-Tac, SL006+pTac-neuB+SL023-Tet were transformed into SLIS026 competent cells, respectively, and the correct clones were screened on LB plates (kanamycin 50 μg/mL, chloramphenicol 25 μg/mL, ampicillin 100 μg/mL) to obtain a strain E. coli SLIS026-6SL carrying 6′-SL synthesis pathway.

### 3.2.2 Production of 6′-SL by SLIS026-6SL Strain

(1) TB medium: 12 g of trypsin, 24 g of yeast extract, 4 mL of glycerol, 2.31 g KH₂PO₄, 12.54 g K₂HPO₄, supplemented to 1000 mL with deionized water, and finally divided into conical flasks at 100 mL/bottle and wrapped well, sterilized at 121° C. for 30 min, and stored at room temperature.

(2) LB medium: 10 g of tryptone, 5 g of yeast extract, 10 g of NaCl, and 15 g of agar were weighed respectively, dissolved with distilled water and mixed well, and were supplemented to 1 L after the pH was adjusted to 7.2 with 1 mol/L NaOH, and finally divided into conical flasks and wrapped well, sterilized at 121° C. for 30 min, and stored at 4° C. The LB liquid was withour agar.

(3) 200 g/L MgSO₄·7H₂O stock solution: 10 g MgSO4·7H₂O solution was weighed and added into deionized water, supplemented to 50 mL after completely dissolved, then sterilized at 121° C. for 30 minutes, and stored at room temperature.

(4) Trace element stock solution: 54.4 g/L ferric ammonium citrate, 9.8 g/L MnCl₂·4H₂O, 1.6 g/L CoCl₂·6H₂O, 1 g/L CuCl₂·2H₂O, 1.9 g/L H₃BO₃, 9 g/L ZnSO₄·7H₂O, 1.1 g/L N₂MoO₄·2H₂O, 1.5 g/L Na₂SeO₃, 1.5 g/L NiSO₄·6H₂O. The trace element stock solution was prepared according to the above concentrations, sterilized at 121° C. for 30 min, and stored at 4° C. for future use.

(5) 1000 g/L glycerol: 1000 g glycerol was weighed, supplemented to 1 L with deionized water, then sterilized at 121° C. for 30 minutes, and stored at room temperature.

(6) 250 g/L lactose: 250 g of lactose was dissolved in deionized water (dissolved by heating), and supplemented to 1 L, then sterilized at 121° C. for 30 min, and stored at room temperature.

(7) The strains were inoculated into 5 mL of LB medium (kanamycin 50 μg/mL, chloramphenicol 25 μg/mL), and cultured at 37° C. and 250 rpm for 4 h . Then the seed liquid was inoculated into fresh TB medium at a ratio of seed liquid: medium=1: 100, cultured at 37° C., 250 rpm to OD600=0.6-0.8, and IPTG was added to a final concentration of 0.1 mM and the medium was cultured under the condition of 25° C., 250 rpm for 15 h for the inducible expression of protein.

(8) After the inducible expression of protein, 1 mL of 200 g/L MgSO₄·7H₂O (final concentration of 2 g/L), 100 μl of trace element stock solution (final concentration of 0.1%), 2 mL of 1000 g/L glycerol (final concentration of 20 g/L) and 2 mL of 250 g/L lactose (final concentration of 5 g/L) were added to the shaking flask, and cultured at 30° C. and 250 rpm for 24 h , and then sampled to detect the content of SL.

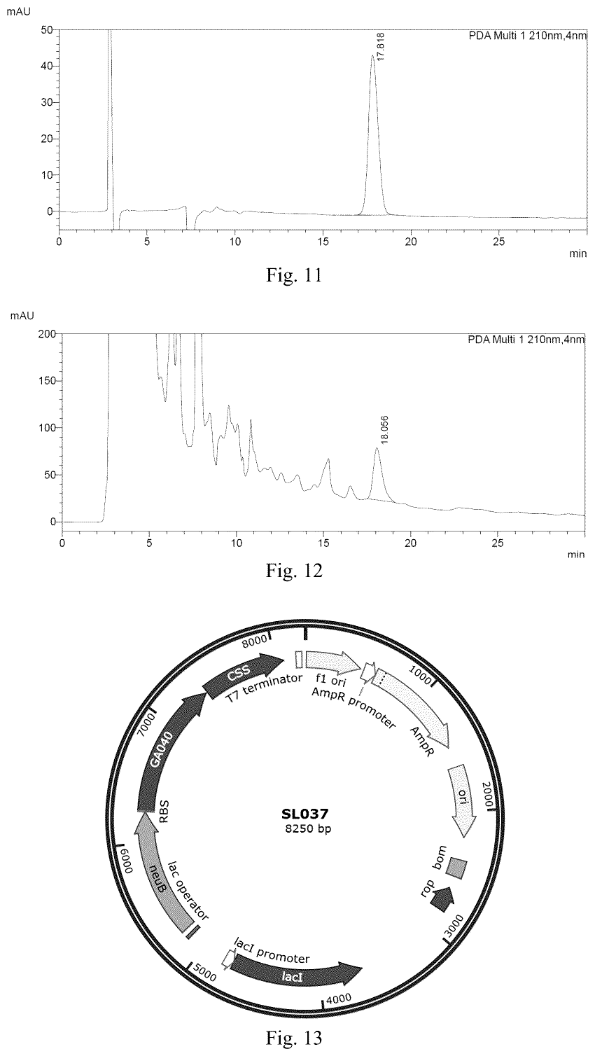

(9) Means of sample treatment: 2-3 mL of the fermentation broth was taken, and the cells were broken by repeated freezing and thawing, placed into boiling water and boiled for 20 minutes after broken, and then centrifuged (4° C., 12000 rpm for 5 minutes) to remove the precipitation and retain the supernatant, and passed through a 0.22 μm filter membrane to detect the content of 6′-SL. The content of 6′-SL in each treatment at 24 h of fermentation is shown in Table 7, when the Tet promoter was used to regulate the expression of neuB, i.e., the combination of SL006+pTet-neuB+SL023, 6′-SL content is the highest, which could reach 2.5 g/L. The SL detection results are shown in the accompanying drawings, FIG. 11 shows the detection profile of 6′-SL standard, and FIG. 12 shows the detection profile of 6′-SL fermentation broth.

TABLE 7
Detection results of 6′-SL content

| | | | 6′-SL |
|---|---|---|---|
| | | | Content |
| | | Colony | (g/L) |
| Host | Treatment | Number | 24 h |
| SLIS026 | SL006 + pET28a − neuB + SL023 | A1 | 0.96 |
| | SL006 + pET28a − neuB + SL023 − Tet | B1 | 0.82 |
| | SL006 + pET28a − neuB + SL023 − Tac | C1 | 0.92 |
| | SL006 + pTet − neuB + SL023 | D1 | 2.51 |
| | SL006 + pTet − neuB + SL023 − Tac | E1 | 1.23 |
| | SL006 + pTet − neuB + SL023 − Tet | F1 | 1.42 |
| | SL006 + pTac − neuB + SL023 | G1 | 1.30 |
| | SL006 + pTac − neuB + SL023 − Tac | H1 | 1.25 |
| | SL006 + pTac − neuB + SL023 − Tet | I1 | 1.32 |

### Example 4 Production of 3′-SL Using SLIS026 Strain

### 4.1 Construction of Expression Plasmids Related to 3′-SL Synthesis

(1) Total gene synthesis was performed on gene SEQ ID NO: 63 encoding α-2,3-sialyltransferase (3ST, GA040) with accession number AJC62560.1, which was synthesized and ligated into the pET28a vector by Sangon Biotech (Shanghai) Co., Ltd.

(2) PCR amplification was performed according to the primers and templates listed in Table 8 to obtain the target fragments. The PCR reaction system and conditions were the same as 1.1 in Example 1.

(3) The amplified DNA fragments were recovered by gel cutting using a gel recovery kit, and positive single colonies were obtained by recombination, transformation, and plate screening using the multi-fragment recombination kit of NEB Company, and then the correct plasmids were obtained by picking up the colony, shaking bacteria, and verifying via sequencing by Tsingke Biotechnology Co., Ltd.

TABLE 8
Primers required for construction of expression plasmid SL037

| | | Primer | | SEQ ID | | Product |
|---|---|---|---|---|---|---|
| Plasmid | Gene | Name | Primer Sequence | NO: | Template | Size |
| SL037 | 3ST | SL37-F1 | tcgcctaaaaggagatataccATGGGCCT | 81 | pET28a- | 1100 bp |
| (FIG. 13) | (GA040) | | GAAAAAAGCGTGCCT | | 3ST | |
| | | SL37-R1 | TTTCCATggtatatctccttTTAGTTT | 82 | | |
| | | | TTATCGTCGAAGGTCAGG | | | |
| | F2 | SL37-F2 | aaggagatataccATGGAAAAACAG | 83 | SL023 | 7.2 kb |
| | | | AACATCGCGGTTATCC | | | |
| | | SL37-R2 | ggtatatctccttttaggcgaaatcttcataagaca | 84 | | |
| | | | gctgc | | | |

### 4.2 Production of 3′-SL During Fermentation

4.2.1 Construction of 3′-SL E. coli Strains

Competent cells were prepared on the basis of the gene knockout strain SLIS026, the specific method was the same as 1.2.1, and then the plasmid combination SL006+pTet-neuB+SL037 was transformed into SLIS026 competent cells with reference to the plasmid combination with the best 6′-SL fermentation result, and the correct clones were screened on LB plates (kanamycin 50 μg/mL, chloramphenicol 25 μg/mL, ampicillin 100 μg/mL) to obtain a strain E. coli SLIS026-3SL carrying the 3′-SL synthesis pathway.

### 4.2.2 Production of 3′-SL by SLIS026-3SL Strain

(1) The type and preparation of the medium required during the experiment are the same as 3.2.

(2) The strains were inoculated into 5 mL of LB medium (kanamycin 50 μg/mL, chloramphenicol 25 μg/mL), and cultured at 37° C. and 250 rpm for 4 h . Then the seed liquid was inoculated into fresh TB medium at a ratio of seed liquid: medium=1: 100, cultured at 37° C., 250 rpm to OD600=0.6-0.8, and IPTG was added to a final concentration of 0.1 mM and the medium was cultured under the condition of 25° C., 250 rpm for 15 h for the inducible expression of protein.

(3) After the inducible expression of protein, 1 mL of 200 g/L MgSO₄·7H₂O (final concentration of 2 g/L), 1 mL of trace element stock solution (final concentration of 0.1%), 2 mL of 1000 g/L glycerol (final concentration of 20 g/L) and 2 mL of 250 g/L lactose (final concentration of 5 g/L) were added to the shaking flask, and cultured at 30° C. and 250 rpm for 24 h , and then sampled to detect the content of SL.

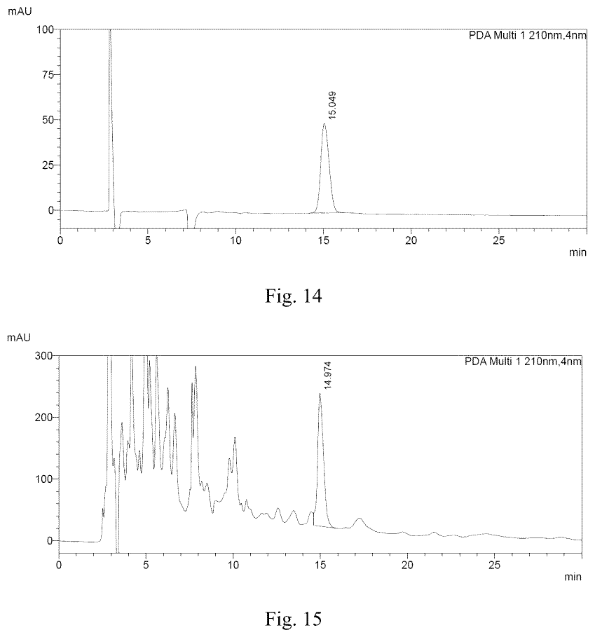

(4) Means of sample treatment: Means of sample treatment were the same as 3.2. Quantitative detection of 3′-SL was carried out, and the results showed that the content of 3′-SL can reach 3 g/L at 24 h ours of fermentation. FIG. 14 shows the detection profile of 3′-SL standard. FIG. 15 shows the detection profile of 3′-SL fermentation broth.

1. A genetically engineered bacterium, wherein the genetically engineered bacterium contains multiple copies of a gene neuB encoding a sialic acid synthase, and the gene neuB is initiated for expression by a Tet promoter; wherein the genetically engineered bacterium further comprises a gene neuB encoding sialic acid synthase, a gene slr1975 encoding N-acetylglucosamine 2-epimerase, a gene YqaB encoding N-acetylglucosamine-6-phosphate phosphatase, a gene Gna1 encoding glucosamine-6-phosphate acetyltransferase, a gene glmS encoding L-glutamine-D-fructose-6-phosphate transaminase, and a gene ppsA encoding phosphoenolpyruvate synthase; the gene neuB, the gene slr1975, the gene YqaB, the gene Gna1, the gene glmS and the gene ppsA are linked in tandem in a plasmid vector 1; the multiple copies are achieved by means of insertion of an exogenous plasmid; the genetically engineered bacterium further comprises a gene encoding N-acylneuraminic acid cytidylyltransferase and a gene encoding a sialyltransferase, the sialyltransferase is α-2,6-sialyltransferase or α-2,3-sialyltransferase.

2. The genetically engineered bacterium of claim 1, wherein an N-acetylneuraminic acid catabolic pathway in the genetically engineered bacterium is disabled.

3. (canceled)

4. The genetically engineered bacterium of claim 1,
wherein, the genes in the plasmid vector 1 meet one or more of the following conditions:
the GenBank accession number of the gene neuB is AF305571;
the GenBank accession number of the gene slr1975 is BAL35720;
the gene YqaB is from the BL21 genome;
the GenBank accession number of the gene Gna1 is NP_116637;
the nucleic acid sequence of the gene glmS is set forth in SEQ ID NO: 66;
the gene ppsA is from the BL21 genome.

5. The genetically engineered bacterium of claim 1, wherein
the gene encoding N-acylneuraminic acid cytidylyltransferase and the gene encoding sialyltransferase are linked in tandem in a plasmid vector 2.

6. The genetically engineered bacterium of claim 1, wherein the starting bacterium is Escherichia coli BL21 (DE3).

7. (canceled)

8. A method for producing sialyllactose by fermentation, comprising using the genetically engineered bacterium of claim 1, adding lactose into a fermentation medium for fermentation, and extracting the sialyllactose from the fermentation broth; and
optionally, obtaining 3′-sialyllactose when the genetically engineered bacterium comprises a α-2,3-sialyltransferase gene; obtaining 6′-sialyllactose when the genetically engineered bacterium comprises α-2,6-sialyltransferase gene.

9. The method of claim 8, wherein the fermentation medium is a TB medium; the TB medium comprises 12 g/L trypsin, 24 g/L yeast extract, 4 mL/L glycerol, 2.31 g/L KH₂PO₄ and 12.54 g/L K₂HPO₄.

10. The method of claim 8, comprising inducing culture with IPTG when cultured to OD value of 0.6 to 0.8;
and/or, supplementing 2 g/L MgSO₄·7H₂O, 20 g/L glycerol, 1 mL/L trace element stock solution and 5 g/L lactose after the induction culture; the trace element stock solution preferably comprises 54.4 g/L ferric ammonium citrate, 9.8 g/L MnCl₂·4H₂O, 1.6 g/L CoCl₂·6H₂O, 1 g/L CuCl₂·2H₂O, 1.9 g/LH₃BO₃, 9 g/L ZnSO₄·7H₂O, 1.1 g/L Na₂MoO₄·2H₂O, 1.5 g/L Na₂SeO3 and 1.5 g/L NiSO₄·6H₂O.

11. The method of claim 8, wherein the fermentation culture condition is culture with shaking at 250 rpm at 30° C.

12. The method of claim 2, wherein all or part of the genes in the N-acetylneuraminic acid catabolic pathway in the genetically engineered bacterium are knocked out; preferably one or more of a gene nanK encoding N-acetylmannosamine kinase, a gene nanE encoding N-acetylmannosamine-6-phosphate epimerase, and a gene nanA encoding N-acetylneuraminic acid aldolase are knocked out;
and/or, a gene LacZ encoding a lactose operon beta-galactosidase in the genetically engineered bacterium is knocked out.

13. The method of claim 4, wherein the plasmid vector 1 is pACYCDuet.

14. The method of claim 5, wherein the N-acylneuraminic acid cytidylyltransferase has the NCBI accession number WP_003512903.1, or comprises the nucleic acid sequence set forth in SEQ ID NO: 61.

15. The method of claim 5, wherein the α-2,6-sialyltransferase is an enzyme with NCBI accession number BAF91416, or comprises the nucleic acid sequence set forth in SEQ ID NO: 62; the α-2,3-sialyltransferase is an enzyme with NCBI accession number AJC62560.1, or comprises the nucleic acid sequence set forth in SEQ ID NO: 63.

16. The method of claim 5, wherein the plasmid vector 2 is pET28a.


SEQUENCE LISTING (84 sequences)

SEQ ID NO: 1 - DNA, 35 bp, synthetic construct
  misc_feature 1..35: note = GA001-P1-F4
  source 1..35: mol_type = other DNA
tgccgaccgtctagagtcgacctgcagaagcttag

SEQ ID NO: 2 - DNA, 45 bp, synthetic construct
  misc_feature 1..45: note = GA001-P1-R4
  source 1..45: mol_type = other DNA
aactggcgttacccaacttaatcactagtattatacctaggactg

SEQ ID NO: 3 - DNA, 47 bp, synthetic construct
  misc_feature 1..47: note = GA001-P1-F1
  source 1..47: mol_type = other DNA
tagtgattaagttgggtaacgccagttttagagctagaaatagcaag

SEQ ID NO: 4 - DNA, 35 bp, synthetic construct
  misc_feature 1..35: note = GA001-P1-R1
  source 1..35: mol_type = other DNA
gttccggaattcaaaaaaagcaccgactcggtgcc

SEQ ID NO: 5 - DNA, 39 bp, synthetic construct
  misc_feature 1..39: note = GA001-LF
  source 1..39: mol_type = other DNA
gctttttttgaattccggaacgggaaggcgactggagtg

SEQ ID NO: 6 - DNA, 37 bp, synthetic construct
  misc_feature 1..37: note = GA001-P1-LR
  source 1..37: mol_type = other DNA
ggtgcgggcctcgacggccagtgaatccgtaatcatg

SEQ ID NO: 7 - DNA, 35 bp, synthetic construct
  misc_feature 1..35: note = GA001-P1-RR
  source 1..35: mol_type = other DNA
tcgactctagacggtcggcaaagaccagaccgttc

SEQ ID NO: 8 - DNA, 32 bp, synthetic construct
  misc_feature 1..32: note = GA001-P1-RF
  source 1..32: mol_type = other DNA
ctggccgtcgaggcccgcaccgatcgcccttc

SEQ ID NO: 9 - DNA, 38 bp, synthetic construct
  misc_feature 1..38: note = pT14-F2
  source 1..38: mol_type = other DNA
gctttttttgaattcgggaattccgcagacaccatctg

SEQ ID NO: 10 - DNA, 35 bp, synthetic construct
  misc_feature 1..35: note = pT-G08-R1
  source 1..35: mol_type = other DNA
ccaaaagttaatgatgggtgaagtacagtcattac

SEQ ID NO: 11 - DNA, 33 bp, synthetic construct
  misc_feature 1..33: note = pT-G08-F2
  source 1..33: mol_type = other DNA
gtacttcacccatcattaacttttggttttgac

SEQ ID NO: 12 - DNA, 33 bp, synthetic construct
  misc_feature 1..33: note = pT-G08-R2
  source 1..33: mol_type = other DNA
gtcgactctagagcgtgcgctgttctttatcgg

SEQ ID NO: 13 - DNA, 31 bp, synthetic construct
  misc_feature 1..31: note = pT-G08-F3
  source 1..31: mol_type = other DNA
cagcgcacgctctagagtcgacctgcagaag

SEQ ID NO: 14 - DNA, 39 bp, synthetic construct
  misc_feature 1..39: note = pT-G08-R3
  source 1..39: mol_type = other DNA
accacgcgtggcttgcagatttactagtattatacctag

SEQ ID NO: 15 - DNA, 41 bp, synthetic construct
  misc_feature 1..41: note = pT-G08-F1
  source 1..41: mol_type = other DNA
agtaaatctgcaagccacgcgtggttttagagctagaaata

SEQ ID NO: 16 - DNA, 45 bp, synthetic construct
  misc_feature 1..45: note = pT4-1R1
  source 1..45: mol_type = other DNA
ctaaaaccgatcgtcgtgaacttcctaactagtattatacctagg

SEQ ID NO: 17 - DNA, 38 bp, synthetic construct
  misc_feature 1..38: note = pT15-F2
  source 1..38: mol_type = other DNA
gctttttttgaattcgcagcgcctgaatcggccatgag

SEQ ID NO: 18 - DNA, 35 bp, synthetic construct
  misc_feature 1..35: note = pT15-1R2
  source 1..35: mol_type = other DNA
gaggtatttgttgtttcccctcgctcgcccctacc

SEQ ID NO: 19 - DNA, 38 bp, synthetic construct
  misc_feature 1..38: note = pT15-1F3
  source 1..38: mol_type = other DNA
gcgaggggaaacaacaaatacctctgaagtgatgcttg

SEQ ID NO: 20 - DNA, 38 bp, synthetic construct
  misc_feature 1..38: note = pT15-R3
  source 1..38: mol_type = other DNA
caggtcgactctagaccgcccgctggcgcgtaaaaaac

SEQ ID NO: 21 - DNA, 45 bp, synthetic construct
  misc_feature 1..45: note = pT15-P1-F1
  source 1..45: mol_type = other DNA
actagtatcgacggtttatacgtggggttttagagctagaaatag

SEQ ID NO: 22 - DNA, 33 bp, synthetic construct
  misc_feature 1..33: note = pT15-R1
  source 1..33: mol_type = other DNA
gcgaattcaaaaaaagcaccgactcggtgccac

SEQ ID NO: 23 - DNA, 34 bp, synthetic construct
  misc_feature 1..34: note = pT15-F4
  source 1..34: mol_type = other DNA
gcgggcggtctagagtcgacctgcagaagcttag

SEQ ID NO: 24 - DNA, 41 bp, synthetic construct
  misc_feature 1..41: note = pT15-1R4
  source 1..41: mol_type = other DNA
aaccccacgtataaaccgtcgatactagtattatacctagg

SEQ ID NO: 25 - DNA, 29 bp, synthetic construct
  misc_feature 1..29: note = Lacz-YZ1-F
  source 1..29: mol_type = other DNA
cgcgctgttagcgggcccattaagttctg

SEQ ID NO: 26 - DNA, 28 bp, synthetic construct
  misc_feature 1..28: note = LAC-YZ2-R
  source 1..28: mol_type = other DNA
ggtcttcatccacgcgcgcgtacatcgg

SEQ ID NO: 27 - DNA, 27 bp, synthetic construct
  misc_feature 1..27: note = nanA-YZ-F1
  source 1..27: mol_type = other DNA
catggtgatgtagcctggcgcaaagcc

SEQ ID NO: 28 - DNA, 28 bp, synthetic construct
  misc_feature 1..28: note = nanA-YZ-R
  source 1..28: mol_type = other DNA
atgggccttatgaacgcatttgattcgc

SEQ ID NO: 29 - DNA, 28 bp, synthetic construct
  misc_feature 1..28: note = nanE-YZ-F
  source 1..28: mol_type = other DNA
gctcgtgcaattccgcttttttctcgac

SEQ ID NO: 30 - DNA, 29 bp, synthetic construct
  misc_feature 1..29: note = nanE-YZ-R
  source 1..29: mol_type = other DNA
cggttatttcgataccgaccagcgtgcag

SEQ ID NO: 31 - DNA, 19 bp, synthetic construct
  misc_feature 1..19: note = CX-targetF-F
  source 1..19: mol_type = other DNA
cagcgagtcagtgagcgag

SEQ ID NO: 32 - DNA, 19 bp, synthetic construct
  misc_feature 1..19: note = CX-targetF-R
  source 1..19: mol_type = other DNA
gacattgcactccaccgct

SEQ ID NO: 33 - DNA, 20 bp, synthetic construct
  misc_feature 1..20: note = Kan-F
  source 1..20: mol_type = other DNA
gaaggagaaaactcaccgag

SEQ ID NO: 34 - DNA, 33 bp, synthetic construct
  misc_feature 1..33: note = Pcr4-R1
  source 1..33: mol_type = other DNA
cagctgcataaaattgcgattggcaaaaccatc

SEQ ID NO: 35 - DNA, 33 bp, synthetic construct
  misc_feature 1..33: note = ACY-GA5-F1
  source 1..33: mol_type = other DNA
aataaggagatataatgaaagaaatcaaaatcc

SEQ ID NO: 36 - DNA, 46 bp, synthetic construct
  misc_feature 1..46: note = ACY-GA5-R
  source 1..46: mol_type = other DNA
ggtgagcgatcatggtatatctccttttaggcgaaatcttcataag

SEQ ID NO: 37 - DNA, 31 bp, synthetic construct
  misc_feature 1..31: note = ACY-GA6-F
  source 1..31: mol_type = other DNA
accatgatcgctcaccgtcgtcaggaactgg

SEQ ID NO: 38 - DNA, 41 bp, synthetic construct
  misc_feature 1..41: note = ACY-GA6-R
  source 1..41: mol_type = other DNA
gctcgtacatggtatatctccttttaagagaccggcagttg

SEQ ID NO: 39 - DNA, 28 bp, synthetic construct
  misc_feature 1..28: note = ACY-GA16-F
  source 1..28: mol_type = other DNA
gatataccatgtacgagcgttatgcagg

SEQ ID NO: 40 - DNA, 45 bp, synthetic construct
  misc_feature 1..45: note = AVY-GA16-R
  source 1..45: mol_type = other DNA
cgggtaagctcatggtatatctcctttcacagcaagcgaacatcc

SEQ ID NO: 41 - DNA, 33 bp, synthetic construct
  misc_feature 1..33: note = ACY-GA9-F
  source 1..33: mol_type = other DNA
taccatgagcttacccgatggattttatataag

SEQ ID NO: 42 - DNA, 42 bp, synthetic construct
  misc_feature 1..42: note = ACY-GA9-R
  source 1..42: mol_type = other DNA
cgataccgcacatggtatatctccttctattttctaatttgc

SEQ ID NO: 43 - DNA, 29 bp, synthetic construct
  misc_feature 1..29: note = ACY-GA10-F
  source 1..29: mol_type = other DNA
taccatgtgcggtatcgttggtgctatcg

SEQ ID NO: 44 - DNA, 48 bp, synthetic construct
  misc_feature 1..48: note = ACY-GA10-R
  source 1..48: mol_type = other DNA
gccattgttggacatggtatatctccttttattccacggtcacggatt

SEQ ID NO: 45 - DNA, 32 bp, synthetic construct
  misc_feature 1..32: note = ACY-GA28-F
  source 1..32: mol_type = other DNA
ccatgtccaacaatggctcgtcaccgctggtg

SEQ ID NO: 46 - DNA, 32 bp, synthetic construct
  misc_feature 1..32: note = ACY-GA28-R
  source 1..32: mol_type = other DNA
ccgcaagcttttatttcttcagttcagccagg

SEQ ID NO: 47 - DNA, 28 bp, synthetic construct
  misc_feature 1..28: note = ACY-F
  source 1..28: mol_type = other DNA
gaaataaaagcttgcggccgcataatgc

SEQ ID NO: 48 - DNA, 36 bp, synthetic construct
  misc_feature 1..36: note = PACY-R
  source 1..36: mol_type = other DNA
gatttctttcattatatctccttattaaagttaaac

SEQ ID NO: 49 - DNA, 34 bp, synthetic construct
  misc_feature 1..34: note = 28a-G5F2
  source 1..34: mol_type = other DNA
gatttcgcctaagcttgcggccgcactcgagcac

SEQ ID NO: 50 - DNA, 34 bp, synthetic construct
  misc_feature 1..34: note = 28a-G5R2
  source 1..34: mol_type = other DNA
gatttctttcatatggctgccgcgcggcaccagg

SEQ ID NO: 51 - DNA, 32 bp, synthetic construct
  misc_feature 1..32: note = 28a-G5F1
  source 1..32: mol_type = other DNA
ctggattttgatttctttcatatggctgccgc

SEQ ID NO: 52 - DNA, 35 bp, synthetic construct
  misc_feature 1..35: note = 28a-G5R1
  source 1..35: mol_type = other DNA
ccgcaagcttaggcgaaatcttcataagacagctg

SEQ ID NO: 53 - DNA, 36 bp, synthetic construct
  misc_feature 1..36: note = Tac-F1
  source 1..36: mol_type = other DNA
gaaatttgacaattaatcatcggctcgtataatgtg

SEQ ID NO: 54 - DNA, 38 bp, synthetic construct
  misc_feature 1..38: note = Tac-R1
  source 1..38: mol_type = other DNA
ggctgctgcccatgtatatctccttcttaaagttaaac

SEQ ID NO: 55 - DNA, 35 bp, synthetic construct
  misc_feature 1..35: note = Tac-F2
  source 1..35: mol_type = other DNA
gaaggagatatacatgggcagcagccatcatcatc

SEQ ID NO: 56 - DNA, 37 bp, synthetic construct
  misc_feature 1..37: note = Tac-R2
  source 1..37: mol_type = other DNA
gatgattaattgtcaaatttcgcgggatcgagatctc

SEQ ID NO: 57 - DNA, 35 bp, synthetic construct
  misc_feature 1..35: note = Tet-F1
  source 1..35: mol_type = other DNA
gatcccgcgaaatgttgacactctatcattgatag

SEQ ID NO: 58 - DNA, 34 bp, synthetic construct
  misc_feature 1..34: note = Tet-R1
  source 1..34: mol_type = other DNA
tgatttctttcatttgtatatctccttcttaaag

SEQ ID NO: 59 - DNA, 33 bp, synthetic construct
  misc_feature 1..33: note = Tet-F2
  source 1..33: mol_type = other DNA
gatatacaaatgaaagaaatcaaaatccagaac

SEQ ID NO: 60 - DNA, 36 bp, synthetic construct
  misc_feature 1..36: note = Tet-R2
  source 1..36: mol_type = other DNA
gatagagtgtcaacatttcgcgggatcgagatctcg

SEQ ID NO: 61 - DNA, 697 bp, synthetic construct
  misc_feature 1..697: note = Optimized nucleotide sequence of N-acylneuraminate cytidylyltransferase(CSS)
  source 1..697: mol_type = other DNA
atgaaaaacatggcgatcatcccggcgcgtgcgggtagcaaaggcctgaaagataaaaacatcaaactgctgaacggcaaaccgatgctggcgtacaccatcgaagcggcgaaagaaagcggcctgttcgaagaaatcatggttagcaccgatagcgaaaaatacgcggaaatcgcgaaacagtggggtgcgagcgttccgttcctgcgtccggttgaactgagcaacgataccgcgagcagctgggatgttgttaaatacgttatcgaaaaatacaaagaactgggccgtgaatttgataccgtggcgctgctgcaaccgaccagcccgctgcgtaacagcgcggacatcatcaaaggttacgaaatcatgaaagaaaaacacgcgaacagcgttatcgcggtttgcgaagcggaacacagcccgctgtggatgaacatcctgccggaaaaccacagcatggcgggcttcatccgtccggatgcggcgaacgtgccgcgtcagagcatcccgacctactaccgtatcaacggcgcgctgtacatcgttaaagtggaatacctgatgaacagcaacgacatctacagcgataaaagctacgcgctgatcatggataaagaaaacagcatcgacatcgatgatatgttcgatttcaaaatcgcgagcctgatcctgcgtgatcgtaaacagtaagctt

SEQ ID NO: 62 - DNA, 1449 bp, synthetic construct
  misc_feature 1..1449: note = Nucleotide sequence of -2,6- sialyltransferase (6ST)
  source 1..1449: mol_type = other DNA
atgaacgataatcaaaatacggtggacgtggtggtctcaaccgtcaacgataacgtgatcgaaaacaacacgtaccaagtcaaaccgatcgataccccgaccacgttcgactcatactcgtggattcagacgtgcggcaccccgatcctgaaagatgacgaaaaatatagcctgtcttttgatttcgttgccccggaactggatcaagacgaaaaattctgtttcgaatttaccggcgatgtggatggtaaacgttatgtgacgcagaccaacctgacggtggttgcaccgaccctggaagtttacgtcgatcatgcttcactgccgtcgctgcagcaactgatgaaaatcatccagcagaaaaacgaatacagccagaatgaacgctttatttcttggggccgtatccgcctgacggaagataacgcggaaaaactgaatgcccatatttatccgctggcaggcaacaataccagccaggaactggtggacgcagttatcgattacgctgactctaaaaaccgtctgaatctggaactgaacacgaataccggccacagtttccgtaacattgcgccgatcctgcgcgccaccagctctaaaaacaacatcctgatctccaacatcaacctgtacgatgacggtagtgctgaatatgtgtccctgtacaactggaaagataccgacaataaatcacagaaactgagtgattcctttctggttctgaaagactacctgaatggcatcagttccgaaaaaccgaacggtatttatagcatctacaattggcatcagctgtatcactcatcgtattacttcctgcgtaaagattacctgacggtggaaaccaaactgcacgacctgcgcgaatatctgggcggttcactgaaacaaatgtcgtgggatacctttagccagctgtctaaaggcgacaaagaactgttcctgaacattgttggttttgatcaggaaaaactgcagcaagaataccagcaaagcgaactgccgaatttcgtctttacgggcaccacgacctgggcaggcggtgaaaccaaagaatattacgctcagcaacaggtgaacgtcgtgaacaatgcgattaatgaaacctctccgtattacctgggccgtgaacatgacctgtttttcaaaggtcacccgcgcggcggtattatcaatgatattatcctgggctcattcaacaatatgattgacatcccggccaaagtttcgtttgaagtcctgatgatgacgggtatgctgccggataccgttggcggtattgcgagcagcctgtattttagtatcccggccgaaaaagtgtccttcattgtttttaccagttccgatacgatcaccgatcgcgaagacgcgctgaaaagtccgctggtccaagtgatgatgaccctgggcattgtgaaagaaaaagatgtgctgttctggtgctaa

SEQ ID NO: 63 - DNA, 1116 bp, synthetic construct
  misc_feature 1..1116: note = Nucleotide sequence of alpha-2,3- sialyltransferase
  source 1..1116: mol_type = other DNA
atgggcctgaaaaaagcgtgcctgaccgttctgtgcctgatcgttttctgcttcggcatcttctacaccttcgatcgcgttaaccagggcgaacgcaacgcggtttctctgctgaaagaaaaactgttcaacgaagaaggtgaaccggttaacctgatcttctgctacaccatcctccagatgaaagttgcggaacgtatcatggcgcagcatccgggcgaacgcttctacgttgtgctgatgagcgaaaaccgcaacgaaaaatacgactactacttcaaccagatcaaagataaagcggaacgtgcgtacttcttccacctgccgtacggtctgaacaaatccttcaacttcatcccgacgatggcggaactgaaagttaaatctatgctgctgccgaaagtgaaacgcatctacctggcgtccctggaaaaagtgagcatcgcggcattcctgtctacctacccggacgcagaaatcaaaaccttcgatgatggcaccggcaacctgatccagtccagctcttacctgggcgacgagttctctgttaacggcaccatcaaacgtaacttcgcacgtatgatgatcggcgattggtccatcgcgaaaacccgtaacgcgtctgatgaacactacaccatcttcaaaggcctgaaaaacatcatggatgatggtcgtcgtaaaatgacctacctgccgctgttcgatgcgtctgaactgaaaaccggtgacgaaaccggcggcaccgttcgtatcctgctgggcagcccggataaagaaatgaaagaaatcagcgaaaaagcggcgaaaaacttcaaaatccagtacgttgcgccgcacccgcgccagacctacggcctgtctggcgttaccactctgaacagcccgtacgtgatcgaagattacatcctgcgtgaaatcaagaaaaacccgcacacccgctacgaaatctacaccttcttcagcggtgcggcgctgaccatgaaagacttcccgaacgttcacgtttacgcgctgaaaccggcgagcctgccggaagattactggctgaaaccggtgtacgcgctgttcacccagagcggcatcccgatcctgaccttcgacgataaaaactaa

SEQ ID NO: 64 - DNA, 119 bp, synthetic construct
  misc_feature 1..119: note = Tac promoter
  source 1..119: mol_type = other DNA
ttgacaattaatcatcggctcgtataatgtgtggaattgtgagcggataacaatttcacacaggaaacagaattgcccctctagaaataattttgtttaactttaagaaggagatatac

SEQ ID NO: 65 - DNA, 124 bp, synthetic construct
  misc_feature 1..124: note = Tet promoter
  source 1..124: mol_type = other DNA
gttgacactctatcattgatagagttattttaccactccctatcagtgatagagaaaagtgaaatgaatagttcgacaaaaatctagaaataattttgtttaactttaagaaggagatatacaa

SEQ ID NO: 66 - DNA, 1830 bp, synthetic construct
  misc_feature 1..1830: note = Glms
  source 1..1830: mol_type = other DNA
atgtgcggtatcgttggtgctatcgcacagcgtgatgtagcgaaaatcctcctggaaggtctgcgtcgtctcgaataccgtggttacgactctgccggtctggcagtagtggatgcagaaggtcacatgactcgtctgcgtcgtctgggtaaagtgcagatgctcgcgcaggcggcggaagaacacccactccacggtggtacgggtatcgcacacactcgttgggcaacccacggtgaaccgtctgaggtcaacgcacacccgcatgttagcgagcacatcgtagtcgttcacaacggtatcatcgagaaccacgaaccactccgtgaggaactcaaagcccgtggttacaccttcgtaagcgaaaccgacacggaagttatcgcccacctcgttaactgggaactcaaacagggtggtactctgcgtgaagcagttctgcgtgccattccacagctgcgtggtgcatacggtaccgtgatcatggactctcgtcatccggataccctgctcgccgcacgttctggttctccactcgttatcggtctgggtatgggtgagaacttcatcgcctctgatcagctggccctgctcccagttacccgtcgcttcatcttcctggaagagggtgacatcgccgaaatcacccgtcgttccgttaacatcttcgacaaaacgggtgcggaagttaaacgtcaggacatcgagtctaacctgcagtatgacgctggtgacaaaggcatctaccgtcactacatgcagaaagagatctacgaacagccgaacgcgatcaaaaacaccctgaccggtcgtatctctcacggtcaggttgacctgtctgagctgggtccaaacgcggacgaactcctgtccaaagtcgagcacatccagatcctggcttgtggtacctcttacaactccggtatggtttctcgttactggttcgaatctctggcaggtatcccatgcgacgttgaaatcgcctccgaattccgttatcgtaaatctgcggtacgtcgtaactccctcatgatcaccctgtctcagtctggtgaaaccgctgatactctggcaggtctgcgtctcagcaaagaactgggttacctgggttctctggccatctgcaacgttccgggttctagcctggttcgtgagtctgtgctggctctgatgaccaacgcgggtacggagatcggtgttgcctctaccaaagcgttcactacccagctcactgtcctgctgatgctggttgccaaactgtctcgtctcaaaggcctcgacgctagcatcgaacacgacatcgtacacggtctgcaggccctcccatctcgtatcgagcagatgctgccgcaggacaaacgtatcgaagcactggcagaagacttcagcgacaaacaccacgcgctgtttctgggtcgtggtgaccagtacccaattgcgctggaaggtgccctgaaactgaaagagatcagctacatccatgcagaggcatacgcagcgggtgagctgaaacatggtccactggccctgatcgacgcagatatgccggttattgtggttgctccgaacaacggcctgctggagaaactgaaatccaacatcgaggaagtacgtgcgcgtggtggtcagctgtacgtgtttgctgaccaggacgcgggtttcgtttccagcgacaacatgcacatcatcgaaatgccgcatgttgaagaggtaatcgcgccaatcttctacaccgtaccgctgcagctgctggcgtaccatgtagccctgatcaaaggtacggacgttgaccagccgcgtaacctggcgaaatccgtgaccgtggaataa

SEQ ID NO: 67 - DNA, 34 bp, synthetic construct
  misc_feature 1..34: note = SL23-F1
  source 1..34: mol_type = other DNA
gatatacatatgaacgataatcaaaatacggtgg

SEQ ID NO: 68 - DNA, 44 bp, synthetic construct
  misc_feature 1..44: note = SL23-R1
  source 1..44: mol_type = other DNA
ccatggtatatctccttttagcaccagaacagcacatctttttc

SEQ ID NO: 69 - DNA, 42 bp, synthetic construct
  misc_feature 1..42: note = SL23-F2
  source 1..42: mol_type = other DNA
gtgctaaaaggagatataccatggaaaaacagaacatcgcgg

SEQ ID NO: 70 - DNA, 39 bp, synthetic construct
  misc_feature 1..39: note = SL023-R2
  source 1..39: mol_type = other DNA
gtgcggccgcaagcttaagattctttgtggttcaggatg

SEQ ID NO: 71 - DNA, 36 bp, synthetic construct
  misc_feature 1..36: note = SL023-F3
  source 1..36: mol_type = other DNA
caaagaatcttaagcttgcggccgcactcgagcacc

SEQ ID NO: 72 - DNA, 34 bp, synthetic construct
  misc_feature 1..34: note = SL23-R3
  source 1..34: mol_type = other DNA
gattatcgttcatatgtatatctccttcttaaag

SEQ ID NO: 73 - DNA, 35 bp, synthetic construct; identical to SEQ ID NO: 57
  misc_feature 1..35: note = Tet-F1
  source 1..35: mol_type = other DNA

SEQ ID NO: 74 - DNA, 35 bp, synthetic construct
  misc_feature 1..35: note = SL023-Tet-R1
  source 1..35: mol_type = other DNA
tgattatcgttcatttgtatatctccttcttaaag

SEQ ID NO: 75 - DNA, 34 bp, synthetic construct
  misc_feature 1..34: note = SL23-Tet-F1
  source 1..34: mol_type = other DNA
gatatacaaatgaacgataatcaaaatacggtgg

SEQ ID NO: 76 - DNA, 36 bp, synthetic construct; identical to SEQ ID NO: 60
  misc_feature 1..36: note = Tet-R2
  source 1..36: mol_type = other DNA

SEQ ID NO: 77 - DNA, 39 bp, synthetic construct
  misc_feature 1..39: note = Tac-F1
  source 1..39: mol_type = other DNA
cgcgaaatttgacaattaatcatcggctcgtataatgtg

SEQ ID NO: 78 - DNA, 33 bp, synthetic construct
  misc_feature 1..33: note = SL023-Tac-R1
  source 1..33: mol_type = other DNA
tgattatcgttcatgtatatctccttcttaaag

SEQ ID NO: 79 - DNA, 34 bp, synthetic construct
  misc_feature 1..34: note = SL23-Tac-F1
  source 1..34: mol_type = other DNA
gagatatacatgaacgataatcaaaatacggtgg

SEQ ID NO: 80 - DNA, 38 bp, synthetic construct
  misc_feature 1..38: note = Tac-R2
  source 1..38: mol_type = other DNA
gatgattaattgtcaaatttcgcgggatcgagatctcg

SEQ ID NO: 81 - DNA, 44 bp, synthetic construct
  misc_feature 1..44: note = SL37-F1
  source 1..44: mol_type = other DNA
tcgcctaaaaggagatataccatgggcctgaaaaaagcgtgcct

SEQ ID NO: 82 - DNA, 45 bp, synthetic construct
  misc_feature 1..45: note = SL37-R1
  source 1..45: mol_type = other DNA
tttccatggtatatctccttttagtttttatcgtcgaaggtcagg

SEQ ID NO: 83 - DNA, 41 bp, synthetic construct
  misc_feature 1..41: note = SL37-F2
  source 1..41: mol_type = other DNA
aaggagatataccatggaaaaacagaacatcgcggttatcc

SEQ ID NO: 84 - DNA, 41 bp, synthetic construct
  misc_feature 1..41: note = SL37-R2
  source 1..41: mol_type = other DNA
ggtatatctccttttaggcgaaatcttcataagacagctgc